(19) United States
(12) Patent Application Publication
(10) Pub. No.: US 2009/0317501 A1
(43) Pub. Date: Dec. 24, 2009

(54) Pinus Pinea Plant Extracts For Treating Osteoporosis and the Extraction Process Thereof

(76) Inventor: Villoo Morawala Patell, Karnataka (IN)

Correspondence Address: Pearl Cohen Zedek Latzer, LLP, 1500 Broadway, 12th Floor, New York, NY, 10036, US

(21) Appl. No.: 12/307,269

(22) PCT Filed: Dec. 27, 2006
(86) PCT No.: PCT/IB2006/003836

§ 371 Date: Jan. 1, 2009

(30) Foreign Application Priority Data
Jul. 3, 2006 (IN) 1142/CHE/2006

(51) Int. Cl. A61K 36/15; G01N 33/567; A61P 19/10

(52) U.S. Cl. 424/770; 435/7.21

(57) ABSTRACT

The present invention is concerned with nutritional or pharmaceutical compositions comprising extracts or concentrates of plants and the mixtures thereof belonging to Pinus. sp with specific reference to Pinus pinea. The extracts have been screened and characterized for their activity in the treatment of increased bone resorption and bone formation, osteoporosis in particular. The invention further relates to the use of the extracts as a supplement or a medicament useful in the treatment/alleviation or prevention of diseases or conditions characterized by increased bone resorption and a method of promoting bone growth, maintenance of bone health, comprising the administration of an therapeutically effective amount of the composition containing the extract.

### FIELD OF THE INVENTION

The present invention is concerned with nutritional or pharmaceutical compositions comprising extracts or concentrates of plants and the mixtures thereof belonging to Pinus. sp with specific reference to Pinus pinea. The extracts have been screened and characterized for their activity in the treatment of increased bone resorption and bone formation, osteoporosis in particular. The invention further relates to the use of the extracts as a supplement or a medicament useful in the treatment/alleviation or prevention of diseases or conditions characterized by increased bone resorption and a method of promoting bone growth, maintenance of bone health, comprising the administration of an therapeutically effective amount of the composition containing the extract.

### BACKGROUND OF THE INVENTION

Bone is under a constant process of resorption and formation. As we age, formation lessens and after a peak bone mass is achieved, bone mass remains stable (resorption and formation are equal). Bone resorption is the gradual loss of bone. Osteoporosis, a disease endemic in Western society, typically reflects an imbalance in skeletal turnover so that bone resorption exceeds bone formation. Bone resorption is the unique function of the osteoclast, and anti-osteoporosis therapy to date has targeted this cell (Science, 2000 Sep. 1; 289(5484):1504-8). Affecting 200 million individuals world-wide, osteoporosis is the most common metabolic bone disorder which leads to an increased level of bone fragility and susceptibility to fracture (Walker-bone, et al. 2002; Lin and Lane, 2004). It has been estimated that one in every two Caucasian women will experience an osteoporotic fracture at some point in their lifetime (Lane et al., 2000). Accelerated bone loss affects more than 25 million Americans, the majority of them are postmenopausal women. But men, younger women, and otherwise healthy, active individuals can also experience the dangerous thinning and weakening of bones associated with osteoporosis. Osteoporosis can be generally defined as the reduction in the quantity of bone, either from the reduction in bone formation or the acceleration of bone resorption, in either event the result is a decrease in the amount of skeletal tissue.

### Classification of Osteoporosis:

Based on its etiology, osteoporosis is categorized as a primary or secondary disease. The latter involves the onset of osteoporosis as a result of an existing condition such as an endocrine disorder, the use of certain medications, a hematopoietic disorder, immobilization, or a nutritional, gastrointestinal or connective tissue disorder (Lin and Lane, 2004). Primary osteoporosis is further subdivided into two types. Type I generally occurs in postmenopausal women and is attributed to loss of gonadal hormone function, such as estrogen deficiency associated with menopause (Lin and Lane, 2004; Simon, 2004). Type II osteoporosis generally called as senile osteoporosis is age-related, affecting both men and women over the age of 60. (Lin and Lane 2004) Through the assessment of bone mineral density (BMD; in g/cm 2) using dual energy x-ray absorptiometry (DEXA), the World Health Organization has defined osteoporosis as a BMD more than 2.5 standard deviations below the mean of normal, healthy individuals at their peak bone mass (Lin and Lane, 2004; Simon, 2004; Christodoulou and Cooper, 2003).

### Etiology of Osteoporosis:

Although numerous risk factors have been identified to increase the likelihood of developing this disease, including Caucasian race, advanced age, female gender, history of fracture, smoking and alcoholism, the exact cause of osteoporosis has not yet been identified. Despite this, numerous theories have been proposed in an attempt to explain its etiology. Some theories regarding the etiology of osteoarthritis include bone cell senescence, lifestyle factors (primarily exercise and nutrition) and loss of vitamin D metabolism with age (Tsai, et al., 1984). The latter hypothesis infers that aging leads to an impaired metabolism of vitamin D. Activated vitamin D is a signaling molecule that is largely involved in the regulation of intestinal calcium absorption Tsai, et al., 1984). Therefore, poor vitamin D metabolism leads to a decrease in intestinal calcium absorption and results in PTH signaling, by the endocrine system, to withdraw calcium from the bones. Over time, this continuous removal of calcium from the bones leads to decreased bone mass and development of osteoporosis.

Another theory has proposed that alterations in the regulation of cellular apoptosis, and therefore an inability to control the lifespan of osteoclasts and osteoblasts, results in the onset of osteoporosis (Weinstein and Manolagas, 1999). In general, it is believed that estrogen deficiency, such as that associated with menopause, results in the inability to induce osteoclast apoptosis and promote osteoblast function during the bone remodeling cycle (Weinstein and Manolagas, 1999). The continuous bone deterioration compromises the integrity of the bone and leads to the onset of osteoporosis.

Finally, a recent theory has emerged which links the onset of osteoporosis to intrauterine programming. Programming is defined as persisting changes in structure and function caused by malnutrition or other factors acting during critical stages in early development (Fall, et al., 1998). It is believed that intrauterine environmental factors, including maternal nutrition and stature, may cause permanent changes to the endocrine system during neonatal development and thus influence both early growth and bone mineralization, as well as adult BMD (Fall, et al., 1998). Specifically, the association between fetal development and adult BMD is thought to be mediated through the intrauterine programming of the growth hormone/insulin growth factor (IGF)-1 and hypothalamic-pituitary-adrenal (HPA) axes (Dennison, et al., 2001). One study has suggested that 62% of the variation in birth weight resulted from the intrauterine environment, 20% resulted from maternal genes and 18% from fetal genes. 14 Thus, by examining birth weight and growth at infancy (markers for intrauterine programming) as well as the levels of systemic signaling factors present in adulthood (regulators of bone turnover), adult bone mass and potential for development of osteoporosis may be predicted (Dennison, et al., 2001).

With considerable impact on such a large number of individuals and, as a result of the high morbidity and mortality associated with osteoporotic related fractures, significant efforts have been made to better understand this disorder. With no cure for this disease, effective treatment methods have been explored to help diminish the risk of disease onset and slow its progression. A successful prevention strategy or cure for osteoporosis will not be attainable until further clarity is reached as to its exact cause. Many theories have been proposed to explain the etiology of this disease and, with numerous studies, insight has been gained as to the origin of this disease.

Conventional treatment methods include biphosphanates (such as aledronate, ibancronate and risedronate, calcitonin, estrogen therapy and hormone therapy and raloxifene) Most of these conventional medications cause side effects such as allergic reactions, nausea, skin rashes, nasal irritations, headaches, breast tenderness, vaginal bleeding, mood disturbances. More specifically with respect to hormone therapy, the Woman's health initiative study recently confirmed that hormone therapy is associated with a modest increase in the risk of breast cancer, strokes and heart attacks. For this reason, The Food and Drug Administration (FDA) recommended that women first considered other osteoporosis medications for prevention and or treatment of osteoporosis. While the existing therapies have some efficacy, there remains a continuing need for alternative therapeutic preferably herbal remedies that can augment or replace the existing therapies. Such herbal medications would be naturally available with minimal or no side effects, lower costs and of equal or greater efficacy than the existing therapies and medications. Nevertheless, it would be preferable instead of treating established osteoporosis with drugs, to prevent increased bone resorption with natural means. Natural means are generally much more acceptable to patients, which results in an increased compliance.

### Bone Formation:

Bone remodeling other than being characterized by osteoclasts, resorption of the pre-existing bone is also followed by de novo bone formation by osteoblasts. Recent evidences have suggested that cells of osteoblastic lineage are involved in osteoclast differentiation and hence there exists a functional link between the two activities, formation and resorption. Functional analyses have showed that in the absence of bone formation, bone resorption continued to occur normally leading to osteoporosis of controlled severity. (David et al, Proceedings of the National Academy of Sciences of the United States of America, Vol 95, 1998). A slow paced bone formation, wherein the new formation is outpaced by bone loss, so that the total skeletal mass slowly declines. Finally since the rate of bone loss accelerates in women at menopause and in both sexes in old age there is a correlation with the onset of post-menopausal osteoporosis in women and age related osteoporosis in both sexes.

The present invention is related to the lead characterization of botanical extracts from Pinus pinea that have been thoroughly analyzed for their potentiality in inhibition of bone resorption and certain extracts for their positive effects on bone formation. Information on 250 plants was collected, studied and analysed for their therapeutic effects on curing of treatment of Osteoporosis related disorders of which 19 different types of plant varieties were identified for their studies based on their anti-osteoporotic activities. Of these 19 species, 18 are of plant origin namely Allium cepa, Allium sativa, Berberis aristata, Camellia sineusis, Carthmus tinctorius, Citrus spp, Eugenia jambolana, Foeniculum vulgare, Glycyrrhiza glabra, Helianthus annuus, Linum usitatissimum, Olea europa, Pinus spp, Prunus spp, Punica granatum, Saccharum officinarum, Salvia officinalis, Sesamum indicum and one is of animal origin namely Coral calyx (Praval Bhasma). The foregoing is only exemplary of plants reported to possess medicinal properties for treatment of osteoporosis and related bone disorders. Pinus pinea has been short listed primarily on account of its proven or suspected anti-oesteoporotic activities, its role as an antioxidant, its activity in reduction of bone resorption or aiding in Calcium uptake. Pinus pinea is also known as Italian stone pine (Rikli 1943), stone pine; Mediterranean stone pine; (obsolete): Umbrella pine. This very distinct pine, has been placed in Sect. Pinea, subsect. Pineae, in which it is the only species; the section also includes subsect. Pinaster, which has similar foliage but very different cones (see e.g. P. pinaster, P. canariensis, P. brutia).

Many pines have been used to produce turpentine, a semi-fluid, yellow or brownish resin (oleoresin). The turpentine obtained from the resin of all pine trees is known for its antiseptic, diuretic, rubefacient and vermifuge properties. It is a valuable remedy used internally in the treatment of kidney and bladder complaints and is used both internally and as a rub and steam bath in the treatment of rheumatic affections. It is also very beneficial to the respiratory system and so is useful in treating diseases of the mucous membranes and respiratory complaints such as coughs, colds, influenza and TB. Externally it is a very beneficial treatment for a variety of skin complaints, wounds, sores, burns, boils etc and is used in the form of liniment plasters, poultices, herbal steam baths and inhalers.

The subject matter of the current invention describes extracts isolated from Pinus pinea which was the result of the planned experiments conducted to test the toxicity of the said extracts, further analysis of their potential positive osteogenic properties which include its potent inhibitory effect on bone resorption and enhancement of bone formation.

This background information is provided for the purpose of making known information believed by the applicant to be of possible relevance to the present invention. No admission is necessarily intended, nor should be construed, that any of the preceding information constitutes prior art against the present invention.

### DESCRIPTION OF THE FIGURES

FIG. 1: Cell viability results

FIG. 2: CTX release assays of plant extracts for the resorption experiment

FIG. 3: Cell viability results of plant extracts for the resorption experiment

FIG. 4: CTX release results for plant extract Pp_Se_Et_{—}01 for lead characterization

FIG. 5: cell viability results for the plant extract Pp_Se_Et_{—}01

FIG. 6: The TRAP activity assay for plant extract Pp_Se_Et_{—}01

FIG. 7: The ALP activity assay of plant extracts for the bone formation experiment

FIG. 8: cell viability results of the plant extracts

### SUMMARY OF THE INVENTION

The primary objective of the present invention is to identify, test, characterize and screen extracts isolated from the plant species of family Pinaceae, preferably of the genus Pinus for their potent inhibitory effects on bone resorption and possible bone formation properties.

A particular embodiment of the invention describes the method of a suitable aqueous or organic solvent-based extraction of a specific therapeutically important phytochemical extract.

In accordance with another aspect of the invention, separate invitro tests are conducted to evaluate the toxicity of the extracts to ascertain the safe practical application of the said extracts. The biotherapeutic potential of the said extracts has been studied and confirmed through standard in vitro cell free and cell based assays.

It is a more specific aspect of the invention to provide a novel method of treating, preventing diseases or conditions characterized by increased bone resorption by administering a therapeutically effective amount of the composition containing the extract to a mammal in need of such therapy.

It is another specific object of the invention to provide a method of promoting bone growth and maintenance of bone health to a mammal in need of such therapy. Preferably the mammal is human.

It is another object of the invention to provide a novel single medicinal extract or a combination of extracts thereof derived from Pinus spp is preferably orally administrable but the invention contemplates topical, intradermal, intramuscular, parenteral or intravenous administrations thereof.

These and other objects of the subject invention will become apparent from the detailed disclosure provided hereinafter.

### DETAILED DESCRIPTION OF THE INVENTION

Plant material suitable for preparation of the plant extract for inclusion of the therapeutic composition of the invention is derived from a potential plant. Plant extracts capable of inhibiting bone resorption and/or enhancing bone formation have been isolated from a variety of plant species as described herein and are suitable candidate extracts for inclusion in the composition of the invention.

In accordance with a further embodiment of the present invention, the potential plant is a member of the family Pinaceae. In another embodiment of the invention, the potential plant is a member of the genus Pinus. It will be readily apparent to one skilled in art that other extracts capable of potential positive osteogenic properties could be isolated using similar techniques from a wide range of plants i.e., potential plants. The potential plants include all species of the family Pinaceae, including terrestrial, aquatic or other plants that can be subjected to standard extraction procedures such as those described herein in order to generate an extract that can be tested for its therapeutic abilities. Extracts demonstrating inhibitory activity on bone resorption and/or having a positive osteogenic effect on osteoblast bone formation are considered to be suitable candidate extracts for use in the therapeutic compositions of the invention.

In one embodiment of the invention, there is provided a process for obtaining a plant extract capable of inhibiting bone resorption activity of osteoclast cells and/or enhancing the bone formation abilities of the osteoblast cells, the process comprising (a) obtaining plant material from one or more plants (b) obtaining an extract from the plant material by contacting the plant material with an aqueous, an ethanolic or an organic solvent, or a combination thereof, thereby providing one or more plant extracts (c) analyzing the plant extracts for toxicity and presence of inhibitory activity against bone resorption and/or enhancing bone formation capabilities and (d) selecting plant extracts having one or both of these activities.

### Extraction of the Plant Material by Solvent Extraction Process:

The plant material employed in the extraction process can be the entire potential plant, or it can be one or more distinct tissues from the plant for example, leaves, seeds, roots, stems, flowers, or various combinations thereof but preferably the seed of the plant. If desired the plant material can be treated prior to extraction, for example, by drying, freezing, lyophilizing, or some combination thereof. If desired, the plant material can be fragmented and/or homogenized by some means such that a greater surface area is presented to the solvent. For example, the plant material can be crushed or sliced mechanically, using a grinder or other device to fragment the plant parts into small pieces or particles, or the plant material can be frozen liquid nitrogen and then crushed or fragmented into smaller pieces.

The solvent used for the extraction process can be aqueous, alcoholic or organic, or a combination thereof. In one embodiment of the present invention, plant material is extracted with an aqueous solvent. Examples of suitable solvents include but are not limited to water, buffers, cell media, dilute acids or bases and the like. In an alternate embodiment of the invention, the plant material is extracted with an alcoholic solvent. Examples of suitable alcoholic solvents include, but are not limited to methanol, ethanol, n-propanol, iso-propanol, 2-butanol, ter-butanol, and combinations thereof.

In an alternate embodiment, plant material is extracted with an organic solvent. Various extraction processes are known in the art and can be employed in the methods of the present invention. The extract is generally produced by contacting the solid plant material with a solvent with adequate mixing and for a period of time sufficient to ensure adequate exposure of the solid plant material to the solvent such that inhibitory activity present in the plant material can be taken up by the solvent.

In accordance with this embodiment, four basic extraction processes are performed in sequence to generate potential extracts A, B, C and D based on the solvents used that being 80% ethanol, Acetone, Hexane and water.

The solvent extraction process may be selected from direct and successive extraction types such as extraction from plant parts in soxhlet apparatus or in flasks at room temperature or at higher temperature with polar and/or non-polar solvent (s). Regardless of the number of extraction processes, each extraction process typically is conducted over a period of time between about 6 hours to 24 hours at room temperature. Adequate contact of the solvent with the plant material can be encouraged by shaking the suspension. The liquid fraction is then separated from the solid (insoluble) matter resulting in the generation of two fractions: a liquid fraction, which is the potential extract, and a solid fraction. Separation of the liquid and solid fractions can be achieved by one or more standard processes known to those skilled in art.

The present invention contemplates the large-scale preparation of the selected plant extracts of the invention. Such extracts can be prepared on a commercial scale by repeating the extraction process that lead to the isolation of the extract of interest. The small-scale extraction procedure can simply be scaled up and additional steps of quality control can be included to ensure reproducible results for the resulting extracts.

Also contemplated by the present invention are the modifications to the small-scale procedure that may be required during the scale up for the industrial level production of the extract. Such modifications may include for example, alterations to the solvent being used or to the extraction procedure per se employed in order to compensate for variations that occur during the scale-up and render the overall procedure more amenable to industrial scale production, or more cost effective. Modifications of this type are standard in the industry and would be readily apparent to those skilled in the art.

In yet another embodiment of the subject invention, concentration of the purified extracts or partially purified extracts by solvent removal from the original extract and/or fractionated extract, and/or purified extract. The techniques of solvent removal are known to those skilled in the art and include, but are not limited to rotary evaporation, distillation (normal and reduced pressure), centrifugal vacuum evaporation (speed vac), and lyophilisation.

The potential extracts obtained thereof are concentrated and solubilised in an appropriate solvent preferably DMSO (Di-methyl Sulphoxide) prior to the conduction of the various test analysis. Examples of various other organic solvents include but are not limited to, di-ethyl ether, hexane, heptane, dichloromethane, ethyl acetate, butyl alcohol, ether, acetone and the combinations thereof.

### Cell Based Toxicity Tests:

In view of the important role played by osteoblasts in regulating growth and in bone remodelling, a series of tests were conducted to evaluate whether the presence of phytoextracts obtained would influence the growth of osteoblastic cells in vitro.

In one embodiment of the present invention study of the in-vitro toxicity was undertaken through a series of tests that are conducted to evaluate the effect of the extracts on the growth and viability of the osteoblastic cells. To this end, mouse osteoblastic cells, MC3T3 (mouse clavarial osteoblast like cells) were seeded at a density of 20,000 cells in 96 well plates and cultured for one day prior to the addition of the plant extracts. The stock solutions of the extracts dissolved in DMSO were diluted to 3 different concentrations. The cells after addition of the extracts are cultured for 3 days and cell viability is measured by a calorimetric based cell viability assay as exemplified in Example 3. Alternatively, as will be readily apparent to one skilled in the art separate cytotoxicity tests, stability tests and the like can be conducted to evaluate the toxicity of the extracts or compositions can be conducted.

Furthermore, also as readily apparent to one skilled in the art, the therapeutic compositions of the invention will need to meet certain criteria in order to be suitable for human or animal use and to meet the regulatory requirements. Thus, once the composition of the invention has been found to be suitable for animal administration, standard in-vivo and in vitro tests can be conducted to determine the information about the metabolism and pharmacokinetics of the compositions, including data on the drug-drug interactions where appropriate, which can be used to design human clinical trials.

The present invention further contemplates that where toxicity is a factor, for example, in patients who cannot tolerate optimal or standard therapeutic dosages, or in cases where the patient's metabolism is compromised sub-optimal doses would be preferred.

### Determination of the Ability of the Plant Extracts to Inhibit Osteoclastic Bone Resorption:

Studies have determined that osteoclasts are largely derived from CD14 positive monocytes (J. Haemotol, 1999 July; 106(1):167-70). Clonal analysis of haematopoietic cells by surface phenotypes has been used to further identify osteoclast precursor by characterizing osteoclast like cells distinct from other haematopoietic progenitors (Lee et.al; 1992b Muguruma & Lee; 1988). Surface phenotype analysis has shown that human osteoclasts are derived from CD14 monocytes (Massey & Flanagan, 1999). CD 14 marker is strongly expressed on monocytes, the putative osteoclast precursor in peripheral blood and CD14 positive monocytes have been selected for osteoclastogenesis. (Clin. Sci (Lond) 2000, August, 99 (2): 133-40).

Determination of the ability of the plant extracts to inhibit osteoclastic bone resorption has been undertaken wherein the potential extracts can be tested for their ability to inhibit these cell activity using a variety of techniques known in art, including, but not limited to, those described herein. In the context of the present invention, a plant extract that decreases the activity of the osteoclast cells by at least 40% in comparison to the control is considered capable of inhibiting osteoclastic bone resorption. Thus, in accordance with one embodiment of the invention there is provided a method of screening of plant extracts suitable for inclusion in the therapeutic compositions, the method comprising (a) providing one or more plant extract isolated with a specific solvent (b) analyzing the one or more extract for their inhibitory activity on bone resorption (c) selecting the extracts that decrease the activity of bone resorption by at least 40%, as plant extracts suitable for inclusion in the therapeutic compositions.

One skilled in art would appreciate that there are a variety of methods and techniques for measuring qualitatively and/or quantitatively the ability of the plant extract to have an inhibitory effect on osteoclastic bone resorption. For example there are currently several assays to measure bone resorption. Organ cultures rely upon the release in vitro of Ca from long bones or calvaria from newborn mice or rats (Raisz, 1963; van der Pluijm et al. 1994; Most et al. 1995). The bones are pre-labeled by incorporation into the pups of 45Ca or [3H] proline injected into the pregnant mother. The release of the isotopes from the cultured bones is measured at the end of the experiment, which can be continued for at least 5 days. This assay has been used frequently, but the cell composition of explants is extremely heterogeneous. Bone particle-based assays: Bone particles, obtained by grinding of 45Ca- or [3H] proline-labeled bones, are cultured with osteoclasts. 45Ca or [3H] release is measured at the end of the cultures (Oreffo et al. 1988). Bone slice assay. This widely used assay is based on the observation that isolated osteoclasts make resorption pits on slices of devitalised dentine or bone (Boyde et al. 1984) and that these pits resemble Howship's lacunae.

Various formats may be used if the potential extracts are to be tested against a specific set of cultured osteoclast cells. The assays may be adapted in order to facilitate the simultaneous testing of many potential extracts. Such techniques are being constantly developed and the use of such techniques to identify the potential extract activity are considered to be within the scope of the present invention.

### Determination of the Ability of the Plant Extracts Isolated to Promote Osteoblastic Bone Formation:

The many and varied osteoblast culture systems that have been developed include cultures containing osteoblast or osteoblast-like cells from different species, bones of different ages, and a variety of anatomical sites and pathological states. Systems have also been developed for specific cell populations, such as osteoprogenitor cells and osteocytes. Several recent articles have also discussed various osteoblast cell culture models and provide some critical commentaries about their use (Marie, 1994; Rodan et al. 1994; Gundle & Beresford, 1995, Parfitt, 1995; Roby, 1995). In the present invention MC3T3 cell line was used. These cells are murine osteogenic mesenchymal precursor cells, which can be differentiated into osteoblasts by ascorbic acid and beta-glycerol phosphate.

Determination of the ability of the plant extracts to promote osteoblastic bone formation has been undertaken wherein the potential extracts can be tested for their ability to promote bone formation using a variety of techniques known in art, including, but not limited to, those described herein. In the context of the present invention, a plant extract that increases activity of the osteoblast cells. Thus, in accordance with one embodiment of the invention there is provided a method of screening of plant extracts suitable for inclusion in the therapeutic compositions, the method comprising (a) providing one or more plant extract isolated with a specific solvent (b) analyzing the one or more extract for their activity on bone formation (c) selecting the extracts that promote the activity of bone formation.

One skilled in art would appreciate that there are a variety of methods and techniques for measuring qualitatively and/or quantitatively the ability of plant extracts to have an effect on osteoblastic bone formation activity. Collagen and DNA synthesis, calcification and bone morphology can be tested in order to assay bone formation in culture. DNA synthesis may be measured by labeling bones with methyl [3H] thymidine for their last 2 h in culture (Gronowicz et al. 1994). The DNA content can be measured by fluorimetry (Labarca & Paigen, 1980). The measurement of Ca in cultured bone is an important indicator of bone formation in vitro. A colorimetric assay with o-cresolphthalein is commonly used to measure calcification in TCA extracts of cultured bones (Gronowicz et al. 1989). Calcein, a fluorescent dye that stains calcium phosphate deposits (Hock et al. 1968), can be used to measure calcification in mineralising cell cultures. Calcification, however, can be increased by bone damage or death (Ramp & Neuman, 1971). Therefore, the bone should also be checked by histological examination. Several histological methods can be used to assess bone morphology (Malluche & Faugere, 1986).

One of the most frequently assayed biochemical markers is alkaline phosphatase, which is simple to measure biochemically. (Sodek & Berkman, 1987). Its expression pattern in osteoblasts (Doty & Schofield, 1976; Stein et al. 1996) and its involvement in mineralisation have been extensively documented (Wuthier & Register, 1984). However, many cell types in bone or marrow stroma contain alkaline phosphatase, such as hypertrophic chondrocytes (Wuthier & Register, 1984) and adipocytes (Beresford et al. 1993), while fibroblastic cells also have a low concentration of this enzyme. The alkaline phosphatase activity can provide a good indicator of osteoblast cells if the cartilage and marrow are removed by dissection. The present invention evaluates the bone formation that has occurred through Alkaline phosphatase assay and the proliferation that has occurred through Alamar blue assay.

Various other in-vitro osteogenic potential assays are being constantly developed and the use of such techniques to identify the potential extract activity are considered to be within the scope of the present invention.

Various cell lines can be used in the above assays. Examples of suitable cell lines such as ST2 (mature monocytes and macrophages capable of differentiating into osteoclasts, MLC-6 (osteoclast like cell line derived from mouse, MC3T3-E1 (mouse calvaria, Sudo et al 1983), MBA-15 (Clonal marrow stromal cell line) and the like. Osteoblast cell lines include 2T3(osteoblast cell line), AHTO, HOBIT cell lines and the like can be used for the cell based assays. These cell lines can be obtained from ATCC or various other commercial sources. The invention premeditates the use of such suitable osteoclast and osteoblast cell lines for conducting the cell based assays.

### Use of the Therapeutic Composition:

The present invention envisages the method of treating osteoporosis and other related diseases thereof by administering an effective amount of the therapeutic composition comprising the single plant extract or the screened plant extracts purified there from in combination. The therapeutic compositions of the invention can be administered alone or in combination with one or more standard anti-osteoporotic therapeutics. The present invention also contemplates the administration of sub-optimal doses of the therapeutic composition, for example, chemotherapeutic drug(s), in combination with the therapeutic composition.

Thus, in one embodiment of the present invention, in order to prepare a therapeutic combination, one or more plant extracts is first selected and then the efficacy of the extract(s) in attenuating bone resorption or promoting bone formation is determined using standard techniques as one of those outlined above. The efficacy of the one or more plant extract alone is then compared to the efficacy of the one or more plant extract in combination with varying amounts of another component i.e., another plant extract. The invention also contemplates the combination the plant extract with another synthetic inhibitor or anti-cancer therapeutic. A combination that demonstrates therapeutic index in comparison to the individual properties is considered to be an effective combination.

For compositions comprising two or more plant extracts, various ratios of the constituent plant extracts are contemplated. By a way of example, for a composition comprising two plant extracts, for example, extract A and extract B, the ratio of extract A to extract B can vary anywhere between 1:99 and 99:1. By “anywhere between 99:1 and 1:99” it is meant that the ratio of the two extracts can be defined by any ratio within this ratio can be between 98:2 and about 1:99 between about 98:2 and 2:98, between 97:3 and 1:99, between 97:3 and 2:98, between 97:3 and 3:97, etc. The present invention contemplates the ratio of the two extracts is between about 90:10 and 10:90, 80:20 and 20:80, 70:30 and 30:70, 60:40 and 40:60 or 50:50. Analogous ratios are contemplated for compositions comprising more than two or more plant extracts.

The formulations of the present invention contain at least an effective amount of the therapeutic composition. The effective amount is considered to be that amount of the composition, in weight percent of the overall formulation, which must be present in order to produce the desired therapeutic effect. As would be apparent to one skilled in art, the effective amount may vary, depending upon, for example the disease to be treated and the form of administration. In general the therapeutic composition will be present in an amount ranging from about 1% to 100% by weight of the formulation, 10% to about 90% by weight of the formulation, 20% to about 80% by weight of the formulation, 30% to 70% by weight of the formulation, from about 40% to 60% by weight of the formulation and about 50% by weight of the formulation.

The composition of the invention can be used for a treatment of a variety of bone disorders. Exemplary turnouts include, but are not limited to osteoporosis, bone loss, bone fracture, glucocorticoid induced osteoporosis, Pagets disease, osteoarthritis, peri-prosthetic osteolysis, cartilage degeneration, osteogenesis imperfecta and the like or a combination thereof comprising one or more of the foregoing disorders.

The present invention contemplates the use of the therapeutic compositions at various stages in the disease development and progression, including in the treatment of early stage, or advanced and/or aggressive stage of osteoporosis or related disorders. The administration of the therapeutic composition comprising the isolated and screened extracts to mammal having an early stage of the disorder can help to attenuate the progression of the disease. Alternatively, the compositions can be administered to delay recurrence or relapse and to cure the subject.

The dosage of the therapeutic composition to be administered is not subject to defined limits, but will usually be an effective amount. However it will be understood that the actual amount of the composition to be administered will be determined by a physician, in the light of the relevant circumstances, including the exact condition to be treated, the chosen route of administration, the actual composition administered, the age, the weight, and the response of the individual patient and the severity of the patient's symptoms. The dosage ranges are not intended to limit the scope of the invention in any way.

### Modes of Administration:

For administration to a mammal, the therapeutic composition can be formulated as a pharmaceutical or naturopathic formulation such as phytoceuticals or nutraceuticals, for oral, topical, rectal or parenteral administration or for administration by inhalation or spray. The phytoceutical or naturopathic formulation may comprise the one or more plant extracts in dosage unit formulations containing the conventional non-toxic physiologically acceptable carriers, adjuvants and vehicles. The term parenteral as used herein includes subcutaneous injections, intravenous, intramuscular, intrathecal, intrasternal injections or infusion techniques.

The pharmaceutical or naturopathic formulations may be in a form suitable for oral use, for example, as tablets, troches, lozenges, aqueous or oily suspensions, dispersible powders or granules, emulsion hard or soft capsules, or syrups or elixirs. The therapeutic compositions of the invention may be formulated as phytoceuticals, or nutraceuticals. Phytoceuticals may optionally comprise other plant-derived components and can therefore be delivered by such non-limiting vehicles as teas, tonics, juices or syrups. Nutraceuticals contemplated by the present invention may provide nutritional and/or supplemental benefits and therefore be delivered, for example as foods, dietary supplements, extracts, beverages or the like. Phytoceutical and nutraceuticals can be administered in accordance with conventional treatment programs and/or may be a part of the dietary or supplemental program.

Formulations intended for oral use may be prepared according to methods known in art for the manufacture of pharmaceutical compositions and may contain one or more agents selected from the group of sweetening agents, flavoring agents, coloring agents and preserving agents in order to provide palatable preparations.

Tablets contain the active ingredient in admixture with suitable non-toxic physiologically acceptable excipients including, for example, inert diluents, such as calcium carbonate, lactose, calcium phosphate or sodium phosphate; granulating and disintegrating agents, such as corn starch, or alginic acid, binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period.

Various additives or carriers can be incorporated into the orally delivered pharmaceutical naturopathic formulations or the invention. Optional additives of the present composition include, without limitation, phospholipids, such as phosphatidyl glycerol, phosphotidyl inositol, phosphotidyl serine, phosphotidyl choline, phosphotidyl ethanolamine as well as phosphatidic acids, ceramide, cerebrosides, sphingomyelins and cardiolipins.

Pharmaceutical or naturopathic formulations for oral use may also be presented as hard gelatin capsules wherein the active ingredient is mixed with an inert solid diluent, for example, calcium carbonate, calcium phosphate or kaolin, or as soft gelatine capsules wherein the active ingredient is mixed with water or an oil based medium such as peanut oil, liquid paraffin or olive oil.

A syrup may be made by adding the active extract to a concentrated, aqueous solution of a sugar, for example sucrose, to which may also be added any necessary ingredients. Such accessory ingredient (s) may include flavorings, an agent to retard crystallisation of the sugar or an agent to increase the solubility of any other ingredients, such as polyhydric alcohol for example glycerol or sorbitol.

Oily suspensions may be formulated by suspending the plant extract(s) in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents and/or flavoring agents may be added to provide palatable oral preparations. These formulations can be preserved by the addition of an anti-oxidant such as ascorbic acid.

Dispersible powders and granules suitable for preparation suitable for an aqueous suspension by the addition of water provide the active ingredient in admixture with dispersing or wetting agent, suspending agent and one or more preservatives. Suitable dispersing or wetting agents and suspending agents, sweetening, flavoring and coloring agents may also be present.

In a further aspect of the invention there is provided a comestible, that is to say, a foodstuff comprising at least an extract of the invention, typically in dried form, such as in a lyophilised form. The skilled addressee will appreciate that such cosmetibles may contain more than one extract of the invention and may be used. Such foodstuffs may be used in a prophylactic manner and may contain further extracts having a similar function to the first added extract or further added extracts may be added that have a different prophylactic function. Thus a foodstuff could either comprise extracts that provide for a comestible having a single functional aspect, or a comestible may have a multi-functional prophylactic effect against two or more disease types. It is thought that a multi-functional role could be assigned to pharmaceutical formulations comprising two or more extracts possessing dissimilar therapeutic or prophylactic properties designed either for prophylaxis or for the treatment of more than one disease(s) in a mammal, particularly in a human.

The type of foodstuff or comestible to which at least an extract of the invention may be added includes any processed food such as confectioneries, baked products including breads such as loafs, and flat breads such as pitta bread, naan bread and the like, cakes, snack foods such as museli bars, compressed dried fruit bars, biscuits, dairy products such as yoghurts, milk and milk-based products such as custards, cream, cheese, butter and crème fraiche, simulated dairy food product such as Elmlea products, fruits and vegetable juices, aerated drinks, such as carbonated soft drinks and non-aerated drinks such as squashes, soya milk, rice milk and coconut milk and the like, pastas, noodles, vegetables, seed and nut oils, fruited oils such as sunflower oil, rapeseed oil, olive oil, walnut, hazelnut, and sesame seed oil and the like, and frozen confectioneries such as ice cream, iced yoghurts and the like.

The invention will now be exemplified with reference to the following Examples section. It is to be understood that the examples are not to be construed as limiting the scope of the invention in any way.

### EXAMPLE I

Prior to the extraction process the plant material was suitably homogenized in a homogeniser alternatively in a pulverizer to get a seed powder of approximately 200 mesh size.

Extraction process: 300 grams of the powdered plant material was weighed into the extractor (Soxhlet extractor body) and covered with cotton at the top making sure that the level of material is below one inch of the vapor inlet tube. 1000 ml of the solvent is added into the round-bottomed flask and placed onto the mantle and a few (3-4) ceramic chips are added into it. 500 ml of the solvent is added over the material to wet it. The extractor is placed on the flask, which is in turn connected with the condenser. Cold water was circulated continuously in the condenser from the tap. The mantle is switched on and the temperature is set to the boiling point of the solvent. The vapors of the solvent were allowed to pass through the inlet of the extractor which get condensed and the condensed (distilled) solvent gets collected in the Extractor body thus extracting the compounds from it. When the extractor is completely filled with the solvent, it was drained in the flask. This process was continuous as long as there was stable heat and water circulation. The extraction was continued for 6 hours, 4-5 cycles per hour. After 6 hours the mantle was switched off and water flow was stopped. After cooling the plant material was removed and the plant material was spread over the filter paper to dry at room temperature overnight. The extract was collected in the flask and concentrated as exemplified in Example 2.

### EXAMPLE 2

The extract that was collected in the flask was concentrated as described herein. The flask containing the extract was fit with the empty soxhlet extractor body that was in turn fitted tightly with the condenser. Continuous water flow and heat was maintained until the solvent from the flask was distilled and collected in the extractor body up to level which was one inch below the inlet. The temperature was reduced to avoid charring as the volume of the solvent reduced in the flask. The distilled solvent collected in the extractor was transferred to the solvent bottles and labeled appropriately. The process was continued until only very little solvent was left in the flask and no charring had occurred. The extract was swirled in the flask and transferred to a bottle or a lyophiliser flask to dry under vacuum.

Little amount of solvent, about 500 ml could be added through the condenser outlet with the help of the funnel to hasten the draining of the extract in to the flasks either in the beginning or when the solvent level is low or after terminating the extraction process. (After switching off the mantle if the level of the solvent in the extractor body is not sufficient to be drained off in to the flask). If more quantity of the same material needs to be extracted, then more than one extractor set can be used at the same time independently. Proper labeling of the extract has to be ensured.

Concentration of the extract has to be undertaken at a lower temperature after the initial distillation to avoid the charring of the extract on the sides of the flask. If the extracts tend to deposit at the sides of the flask, the flask is swirled to dissolve the extract when the extractor body is emptied.

Storage and labeling of the extract was done to obtain the Extract ID. The Extract ID contains the first two letters of the generic name and species name of the plant followed by the part of the plant used. This is followed by the first two letters of the solvent used for the extraction and finally the subsequent serial numbers in a consequent fashion for the identification of the four extracts which are the subject matter of the invention.

For Example: Pp_Se_Et_{—}01

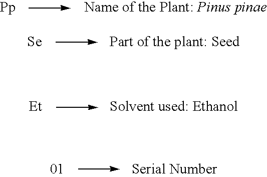

Four different extracts from the plant Pinus pinae were fractionated using four different solvents. The solvents that were used for the extraction were 80% ethanol, acetone, hexane and water. Table 1 refers to the four extracts that were obtained from extraction using the corresponding solvents.

TABLE 1
Extract ID and the solvents used for the extraction process:

| | ID name as referred in the | Solvent used |
|---|---|---|
| Extract ID | figures (screening name) | for extraction |
| Pp_Se_Et_01 | AN04 | 80% Ethanol |
| Pp_Se_Ac_02 | AN10 | Acetone |
| Pp_Se_He_03 | AN22 | Hexane |
| Pp_Se_Wa_04 | AN23 | Water |

50 mg of each extract was weighed out and dissolved in 300 μl of DMSO (dimethyl sulfoxide) prior to analysis by enzymatic assay.

TABLE 2
Extract Ids and the concentration of the extracts:

| Extract ID | 50 mg extract dissolved in | Concentration |
|---|---|---|
| Pp_Se_Et_01 | 300 μl | 167 μg/μl |
| Pp_Se_Ac_02 | 300 μl | 167 μg/μl |
| Pp_Se_He_03 | 300 μl | 167 μg/μl |
| Pp_Se_Wa_04 | 300 μl | 167 μg/μl |

### EXAMPLE 3

The plant extracts were tested in three concentrations to examine the toxicity of the extracts in the MC3T3 cells (mouse calvarial osteoblast like cells) The stock solution of the extracts was diluted 1:100, 1:1000, and 1:10000 in the medium for the cells. The concentrations have been chosen so that the concentrations of DMSO is not higher than 1%. DMSO was added as a negative control in the same concentrations as the plant extracts.

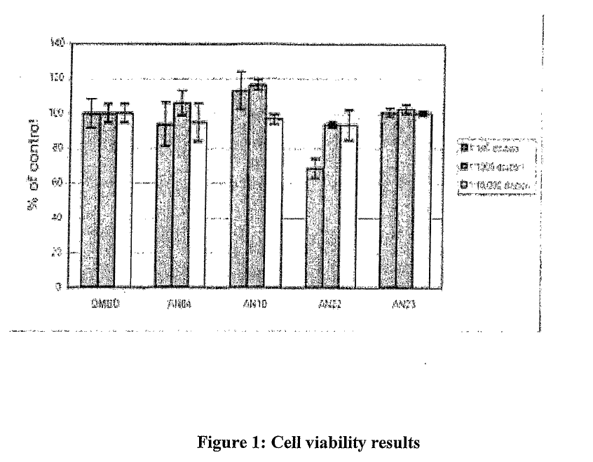

The MC3T3 cells were seeded at a density of 20,000 cells per well in 96 well plates. The cells were cultured for one day before the plant extracts were added in three dilutions. The cells were after the addition of the extracts cultured for three days. At the end of the culture period the cell viability was measured by the Alamar blue assay. The result of the toxicity tests have been represented in the FIG. 1.

Extract Ids Pp_Se_Et_{—}01, Pp_Se_Ac_{—}02 and Pp_Se_Wa_{—}04 were not toxic at any of the dilutions, where as Pp_Se_He_{—}03 was a little toxic at the 1:100 dilution. Dilution 1:10000 and 1:10,000 was used for the screening assays in the cone resorption and the bone formation assays.

### EXAMPLE 4

### Screening Extracts for Bone Resorption Activity

The osteoclasts were isolated as CD 14 positive monocytes and were differentiated into osteoclasts for 10-12 days. Hereafter the osteoclasts were lifted by trypsin, counted and seeded on bovine bone slices at a density of 40,000 cells per bone slice. The osteoclasts were seeded on the cone slices for one day before the plant extracts were added. The osteoclasts were hereby allowed to attach and initiate the resorption activity. The plant extracts were added in two concentrations 1:1000 and 1:10000 dilutions because the toxicity study in the MC3T3 had shown that these concentrations were not toxic to the cells. On each plate were a negative control (DMSO) and a positive control (Bafilomycin)

Bafilomycin is a V-ATPase blocker and it inhibits the osteoclastic bone resorption approximately 8-% compared to DMSO (the negative control was set to 100% on the chart)

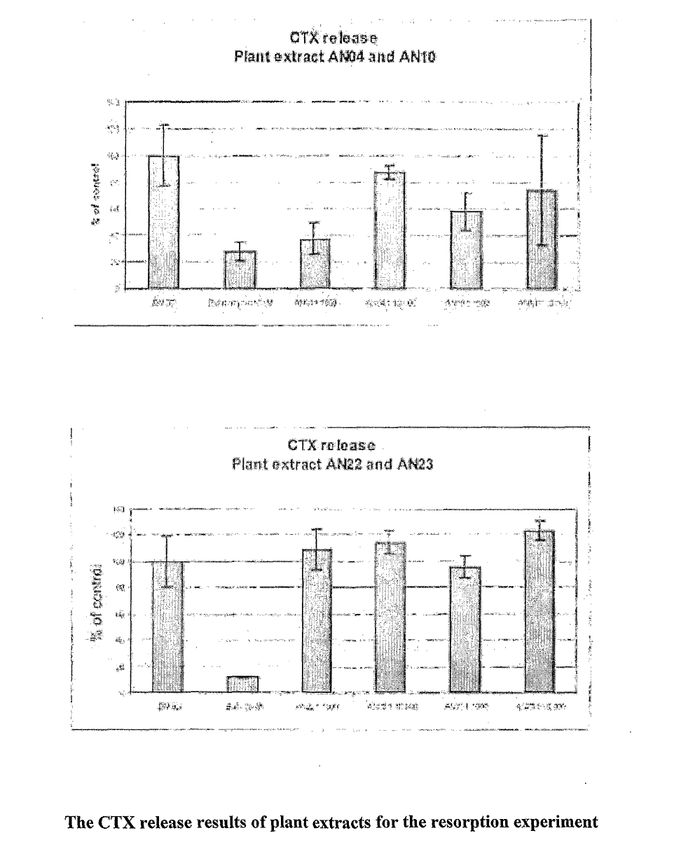

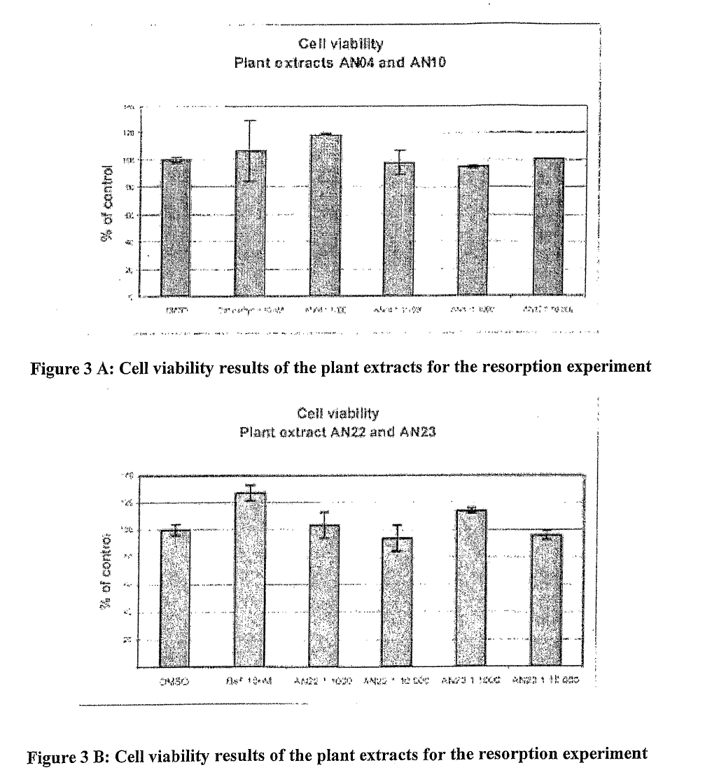

The bone resorption experiment was ended after 5 days of incubation and the cell viability was measured by the use of the Alamar blue assay as represented in FIG. 3. Bone resorption was evaluated as the determination of the CTX release shown in FIG. 2. CTX is the C-terminal type 1 collagen fragments which is released to the culture medium during the osteoclastic bone resorption. The CTX measurement was done according the manufactory instructions.

The plant extract Pp_Se_Et_{—}01, which was the Pinus pinae extract made with 80% ethanol, is not toxic at either dilutions. However, the extract has an inhibitory effect on the bone resorption at the dilution 1:1000. Pp_Se_Et_{—}01 inhibit bone resorption is approximately 60% compared to the control.

The plant extract Pp_Se_Ac_{—}02 was not toxic at either dilution. It has a small inhibition of the resorption at 1:1000, which is approximately 40% compared to the control.

Plant extract Pp_Se_He_{—}03 which is the plant extract made with hexane had no effect on bone resorption.

Plant extract Pp_Se_Wa_{—}04 will be tested further in bone resorption lead characterization, because it inhibited bone resorption up to 60% when compared to the control at the dilution of 1:1000.

### EXAMPLE 5

The osteoclasts were isolated as CD14 positive monocytes and differentiated into osteoclasts for 10-12 days. Hereafter, the osteoclasts were lifted by trypsin, counted and seeded on bovine bone slices for one say before the plant extracts were added. The osteoclasts were seeded on the bone slices for one day before the plant extracts were added. The osteoclasts were hereby allowed to attach and initiate resorption. The plant extracts were added in five dilutions: 1:100, 1:1000, and 1:10000 and 1:100,000 and 1:1000000. On each plate were a negative control, DMSO (1:1000 and 1:10000 dilution) and a positive control, bafilomycin.

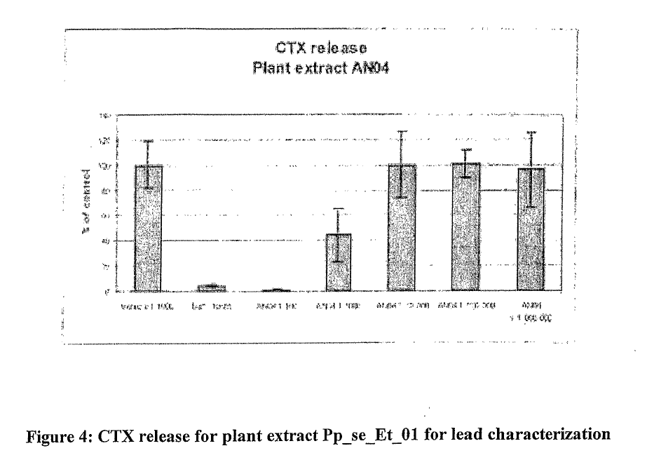

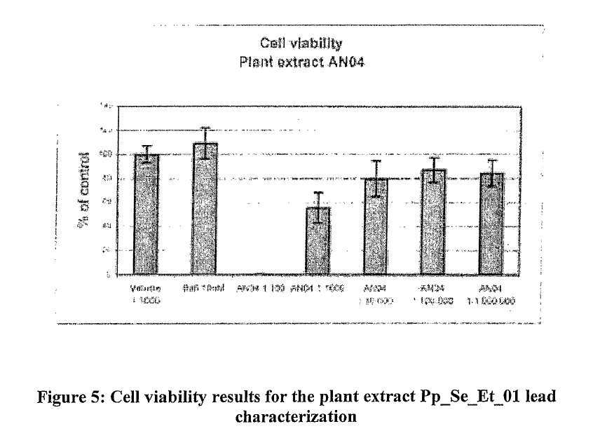

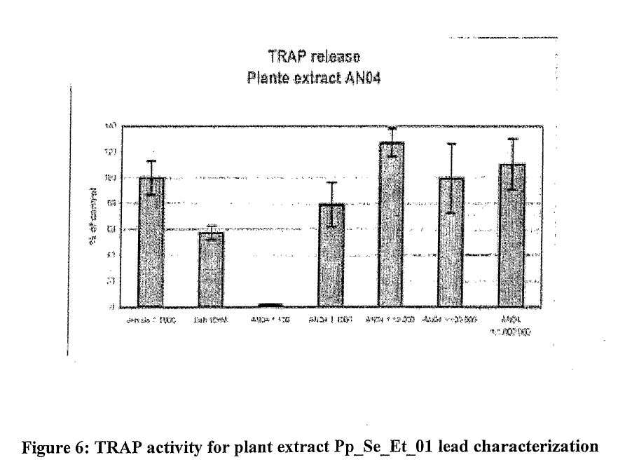

The bone resorption experiment was ended after 10 days of incubation and the cell viability was measured by the use of the Alamar blue assay (FIG. 5). Bone resorption was evaluated as the determination of the CTX release (FIG. 4). Furthermore, the osteoclastic marker marker TRAP was measured (FIG. 6)

The result for the lead characterization of plant extract Pp_Se_Et_{—}01 gave the same result as the hit screening. The inhibitor effect of the extracts on bone resorption was reproduced by the lead characterization. Extract Pp_Se_Et_{—}01 inhibited resorption by 60% compared to the control at dilution 1:1000. The extract was a little toxic at this dilution, but not to the same level as the inhibition of the bone resorption. In the hit screening extract Pp_Se_Et_{—}01 was not toxic to the osteoclasts.

### EXAMPLE 6

### Bone Formation Studies

MC3T3 cell lines were used for the bone formation studies. These cells are murine osteogenic mesenchymal precursor cells, which can be differentiated into osteoclasts by ascorbic acid and beta-glycerol phosphate.

The osteoblasts (MC3T3 cells) were seeded at a density of 75000 cells per well in 24 plates. The cells were culture for one day before the plant extracts were added. Then the osteoblasts were cultured for 14 days in medium supplemented with ascorbic acid and beta-glycerol phosphate (AA and bG) and in the presence of the plant extracts (1:1000 and 1:10000 dilution)

On each plate were a negative control and a positive control added. Medium added without ascorbic acid and beta-glycerol phosphate (without AA and bG) was used as a negative control and this condition does not promoted differentiation of osteoblasts. A positive control was 30 ng/ml BMP-2 used (BMP-2=bone morphogenic protein-2), which induces the differentiation of the osteoblasts.

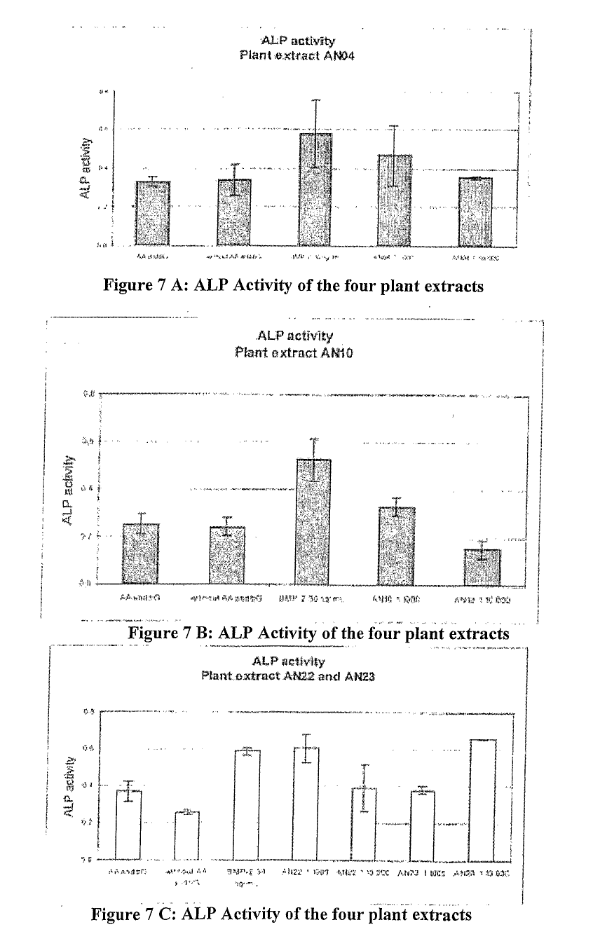

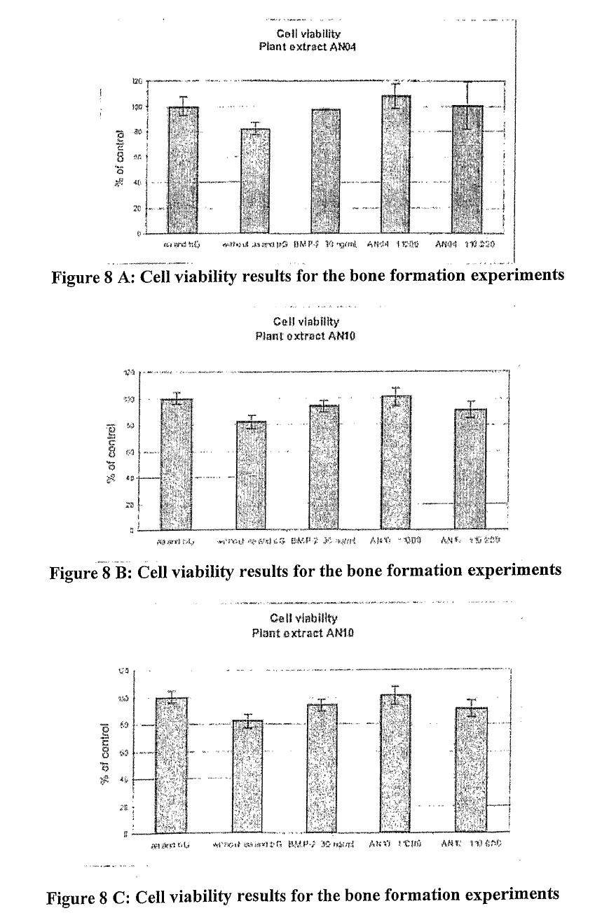

At the end of the culture period, osteoblast bone formation was evaluated by alkaline phosphatase assay (ALP assay) FIG. 7 and the proliferation by Alamar Blue assay (FIG. 8)

For the bone formation experiment BMP-2 is used as a positive control, while the treatment without ascorbic avid and beta-glycerol phosphate (without AA and bG) is the negative control. BMP-2 induces the MC3T3 precursor cells to differentiate into osteoblasts and the ALP activity is induced compared to the negative control.

From measurement of ALP activity it can be concluded that plant extract Pp_Se_He_{—}03 and Pp_Se_Wa_{—}04 has a positive osteogenic effect on the MC3T3 cells. Both the extracts induces the ALP activity to the same level as the positive control BMP-2. Furthermore, the extracts are not toxic to the cells at the used dilutions.

Extract Pp_Se_Et_{—}01 can have a positive effect on the positive effect on the ALP activity at dilution 1:1000.

The extract Pp_Se_Et_{—}01 from Pinus pinae has an effect on bone resorption. Furthermore Pp_Se_He_{—}03 and Pp_Se_Wa_{—}04 has an effect on ALP activity in the bone formation hit screening. Extract Pp_Se_Et_{—}01 has a possible effect on bone formation lead characterization too.

1. A method of treating osteoporosis or conditions related thereof, which are characterized by increased bone resorption in a mammal, which comprises administering to the said mammal a therapeutically effective non-toxic amount of an extract derived from Pinus plant species to decrease bone resorption activity and/or enhancing bone formation capabilities.

2. A method of obtaining a plant extract capable of delaying of onset and/or management of diabetes comprising the following steps:
(a) Obtaining plant material from one or more parts of the plants of claim 1;
(b) Obtaining an extract from the plant material by contacting the plant material with an aqueous, an ethanolic or an organic solvent, or a combination thereof, thereby providing one or more plant extracts;
(c) Analyzing the plant extracts for toxicity and presence of inhibitory activity against bone resorption and/or enhancing bone formation capabilities;
(d) Selecting plant extracts having one or both of these activities.

3. A method according to claim 1, wherein the plant extracts are derived from the plants as set forth in table 1.

4. A method according to claim 1, wherein the plant extracts are derived from the plant of the genus Pinus.

5. A method according to claim 1 wherein, the plant extracts are extracted from leaves, seeds, roots, stems, flowers, or various combinations thereof but preferably the seed of the plant.

6. A method according to claim 1 wherein the method of analyzing the effect on bone resorption is performed invitro and comprises the steps of:
(a) Culturing and exposing the osteoclast cells to the test extract;
(b) Determining the effect of the test extract on bone resorption activity of the osteoclast cells indicative of the extract being useful for the treatment of an osteoporotic disorders.

7. A method according to claim 6, wherein the extracts Pp_Se_Et_{—}01 and Pp_Se_Ac_{—}02 possess an inhibitory effect on bone resorption.

8. The plant extracts of claim 7.

9. A method according to claim 1, wherein the method of analyzing the effect on bone formation is performed invitro and comprises the following steps:
(a) Culturing and exposing the osteoblast cells to the test extract;
(b) Determining the effect of the test extract on bone formation activity of the osteoblast cells indicative of the extract being useful for the treatment of osteoporosis related disorders.

10. A method according to claim 6, wherein the extracts Pp_Se_Et_{—}01, Pp_Se_He_{—}03 and Pp_Se_Wa_{—}04 possess a positive effect on bone resorption.

11. The plant extracts of claim 10.

12. A composition comprising one or more plant extracts are capable of inhibiting osteoclastic bone resorption activity and/or enhancing osteoclastic bone formation.

13. A composition according to claim 12 comprising one or more plant extracts, wherein said composition comprises plant extracts are derived from plants set forth in Table 1.

14. A composition according to claim 12 comprising one or more plant extracts, wherein the plant extracts are derived from a plant belonging to the genus Pinus.

15. A composition according to claim 12 comprising one or more plant extracts, wherein the plant extracts can be used for the treatment or prophylaxis of a disease condition involving increased bone resorption such as Osteoporosis or other related disorders such as bone loss, bone fracture, glucocorticoid induced osteoporosis, Pagets disease, osteoarthritis, peri-prosthetic osteolysis, cartilage degeneration, osteogenesis imperfecta and the like or a combination thereof comprising one or more of the foregoing disorders.

16. A composition according to claim 12 comprising one or more plant extracts, wherein the administration of the therapeutic composition can be formulated as pharmaceutical or naturopathic formulation such as phytoceuticals or nutraceuticals, for oral, topical, rectal, parenteral administration or for administration by inhalation or spray.

17. A composition according to claim 12 comprising one or more plant extracts as set forth in table 1 and a pharmaceutically acceptable carrier.

18. A composition according to claim 12 comprising one or more plant extracts, wherein the pharmaceutical or naturopathic formulations may be in a form suitable for oral use, for example, as tablets, troches, lozenges, aqueous or oily suspensions, dispersible powders or granules, emulsion hard or soft capsules, or syrups or elixirs.

19. A composition according to claim 12 comprising one or more plant extracts, wherein the extracts can be formulated as phytoceuticals or nutraceuticals not limiting to teas, tonics, juices, syrups and nutraceuticals providing nutritional benefits in the form of foods, beverages, supplements and the like.
